(19) United States
(12) Patent Application Publication
(10) Pub. No.: US 2019/0167945 A1
(43) Pub. Date: Jun. 6, 2019

(54) PERMEABLE STRUCTURAL INTERFACE FOR THE CONSTRUCTION OF THIN-WALLED BALLOON CATHETERS

(71) Applicant: Cognition Medical Corp, Atlanta, GA (US)

(72) Inventor: Alexis S. TURJMAN, Cambridge, MA (US)

(73) Assignee: Cognition Medical Corp, Atlanta, GA (US)

(21) Appl. No.: 16/265,692

(22) Filed: Feb. 1, 2019

Related U.S. Application Data
(63) Continuation of application No. PCT/US2017/046493, filed on Aug. 11, 2017.
(60) Provisional application No. 62/374,374, filed on Aug. 12, 2016.

(51) Int. Cl. A61M 25/00

(52) U.S. Cl. CPC A61M 25/0043; A61M 25/0032; A61M 2025/0039; A61M 2025/1061; A61M 2025/0059; A61M 2025/006; A61M 25/005

(57) ABSTRACT

The present invention relates to the construction of thin-walled balloon catheters where navigability, low cross-sectional profile, and low inflation and deflation times are desired. Structural reinforcement of inflation lumen utilizes a soft, flexible, permeable material to fill the fluid channel to maintain patency of the lumen.

### CROSS-REFERENCE TO RELATED APPLICATIONS

This application is a continuation of PCT/US2017/046493 filed Aug. 11, 2017, which claims the benefit of priority to U.S. Prov. App. 62/374,374 filed Aug. 12, 2016, each of which is incorporated herein by reference in its entirety.

### FIELD OF THE INVENTION

The invention relates generally to methods and apparatus for forming and maintaining lumens within a catheter. More specifically, the present disclosure relates to methods and apparatus for forming and maintaining fluid lumens within thin-walled balloon catheters.

### BACKGROUND OF THE INVENTION

Ischemia, the restriction of blood supply to tissue, may result in tissue damage in a process known as ischemic cascade. Damage includes, but is not limited to, shortage of metabolic requirements (i.e., oxygen and glucose), build-up of metabolic waste products, inability to maintain cell membranes, mitochondrial damage, and eventual leakage of autolysing proteolytic enzymes into the cell and surrounding tissues. Brain ischemia may be chronic, e.g., leading to vascular dementia, or acute, e.g., causing a stroke. A stroke is the rapid decline of brain function due to a disturbance in the supply of blood to the brain caused by an obstruction or hemorrhage in a blood vessel. Obstructions encompass emboli, thrombi, and/or thromboemboli. An ischemic stroke is a stroke in which a blood vessel is restricted or occluded by an obstruction.

Devices for treating ischemia, as well as other conditions within the vasculature, may be accomplished using catheter devices which are designed to access the vascular system of patients. Thus, the catheter devices need to remain relatively flexible but when catheter devices are used to access tortuous regions of the vasculature, especially when the catheters are thin-walled micro-catheters, the catheters have a tendency to kink and/or collapse when pushed or torqued from their proximal ends.

This kinking or collapsing not only inhibits the transmission of a push force or torque to the distal end of the catheter, but it also inhibits the flow of fluids or other treatment agents through one or more lumens defined through the length of the catheter.

Therefore, there is a need for methods and devices designed to maintain the patency of one or more lumens defined along or through catheter devices despite the formation of kinks and/or collapse of the catheter.

### SUMMARY OF THE INVENTION

The present invention relates to a novel architecture for the construction of thin-walled balloon catheters. Balloon catheters are used in a wide variety of medical procedures, especially in interventional radiology for the treatment of diseases including cerebral infarction (also known as stroke), peripheral artery disease (PAD), deep vein thrombosis (DVT), pulmonary embolism, abdominal aortic aneurysms (AAA) and acute limb ischemia. In most applications, it is desired to combine navigability, a low cross-sectional profile and low inflation and deflation times.

Common designs for making balloon catheters include, for example, mounting two coaxial cannula such that a fluid channel is created between the external surface of the smaller cannula and the inner surface of the bigger cannula, or extruding multi-lumen cannulas, where one or more lumen are used to fluidly couple a proximal end of the aforementioned lumen and an occlusion membrane like a balloon. As pressure of the inflation fluid is varied, the balloon can be either inflated or deflated. Both of these designs suffer from the probability of occlusion of the lumen carrying the inflation fluid as the catheter is bent rendering the device inoperable. Structural reinforcement of the wall of either or both cannula can remedy to the issue but invariably increase the size of the cross-sectional profile. The present invention proposes to tackle both issue by using a soft, flexible, permeable material to fill the fluid channel dedicated to the operation of one or more balloons.

### BRIEF DESCRIPTION OF THE DRAWINGS

FIG. 1 illustrates one example of accessing the cerebrovasculature by accessing the vasculature via a femoral artery to treat a clot.

FIG. 2 illustrates a detail example of the tortuous nature of the cerebrovasculature in approaching a clot using a treatment catheter and methods as described.

FIGS. 3A and 3B illustrate cross-sectional end views of conventional fluid lumens typically found in catheter devices.

FIGS. 4A and 4B illustrate a side view and a cross-sectional end view of a catheter device having a preamble structural interface which maintains a fluid path through the catheter.

FIGS. 4C and 4D illustrate side and detailed cross-sectional end views of another embodiment of the catheter having a non-axisymmetric shape for the permeable interface.

FIGS. 4E to 4G show yet another embodiment in the side and cross-sectional end views of a hybrid architecture comprised of a first part having coaxial cannulas with an inner cannula and an outer cannula defining a secondary lumen between and a second part that defines an annular orthogonal cross-section.

FIGS. 4H to 4J show yet another embodiment with a hybrid architecture comprised of a first part having a length having the inner cannula and outer cannula disposed such that they define a secondary lumen with an arcuate orthogonal cross-section with respect to the axis of the catheter's primary lumen and a second part that defines an arcuate orthogonal cross-section.

FIGS. 5A and 5E illustrate detail perspective views of a catheter in a straightened configuration and a bent or torqued configuration for comparison purposes.

FIGS. 5B and 5F illustrate cross-sectional end views of a catheter having an inner lumen and an outer lumen which form a fluid lumen in-between when in a straightened and collapsed configuration for comparison purposes.

FIGS. 5C and 5G illustrate cross-sectional end views of a catheter having a fluid lumen formed integrally within the lumen wall when in a straightened and collapsed configuration for comparison purposes.

FIGS. 5D and 5H illustrate cross-sectional end views of a catheter having an inner lumen and an outer lumen which form a fluid lumen in-between where patency of the fluid lumen is maintained by the preamble structural interface despite the catheter being bent or torqued.

FIGS. 6A and 6B illustrate the respective diameters of a conventional catheter and a catheter as described herein.

FIG. 6C illustrates a comparison of the diameters of the conventional catheter and the catheter as described herein to show the reduction of the diameter from the conventional catheter.

FIG. 7 illustrates a perspective and detail partial cross-sectional side view of an interface embodiment having an array of conical micro-pillars for maintaining the fluid lumen.

FIG. 8 illustrates a perspective and detail partial cross-sectional side view of an interface embodiment having a braided interface for maintaining the fluid lumen.

FIGS. 9A to 9C illustrate cross-sectional side and end views of various embodiments where the fluid lumen may be open at a distal opening or along a side portion of the fluid lumen.

FIGS. 10A and 10B illustrate cross-sectional side and end views of an alternative arrangement of the lumen, permeable interface, and balloon.

FIGS. 11A and 11B illustrate a top view of an outer surface of a primary lumen wall splayed out illustrating how the density of micro-pillars may be varied along a length of the interface.

FIGS. 11C and 11D illustrate detail side views of various configurations of the micro-pillar arrays having varying heights.

FIGS. 12A to 12C illustrate perspective and top views of yet another embodiment of the micro-pillar configuration as cylindrical structures.

FIGS. 12D and 12E illustrate top and perspective views of yet another embodiment of the micro-pillar configuration as rectangular structures.

FIGS. 12F and 12G illustrate top and perspective views of yet another embodiment of the micro-pillar configuration as diamond-shaped structures.

FIGS. 12H and 12I illustrate top and perspective views of yet another embodiment of the micro-pillar configuration as elongated diamond-shaped structures.

FIGS. 12J and 12K illustrate top and perspective views of yet another embodiment of the micro-pillar configuration as elliptically-shaped structures.

FIGS. 13A and 13B illustrate top and perspective views of yet another embodiment of the micro-pillar configuration as conically-shaped structures.

FIGS. 13C and 13D illustrate top and perspective views of yet another embodiment of the micro-pillar configuration as truncated pyramid-shaped structures.

FIGS. 13E and 13F illustrate top and perspective views of yet another embodiment of the micro-pillar configuration as truncated diamond-shaped structures.

FIGS. 14A and 14B illustrate top and perspective views of yet another embodiment of the micro-pillar configuration as hemispherically-shaped structures.

FIGS. 15A to 15D illustrate perspective, splayed, and cross-sectional views of another embodiment where a micro-pillar array may be formed along an inner wall of a secondary lumen.

### DETAILED DESCRIPTION OF THE INVENTION

Methods and apparatus for treating patients suffering from cardiovascular maladies encompassing, e.g., embolisms and thromboembolisms, may be used to treat patients by inserting a catheter device into the patient and advancing the catheter through the vasculature in proximity to a treatment site. For instance, a balloon catheter may be used to dilate the vessel, e.g., via balloon inflation, and/or optionally to slightly dilate the vessel to help dislodge clots trapped within the vessel lumen.

While examples and measurements relating to the use of the devices and methods herein may be described in relation to the treatment of, e.g., acute ischemic stroke, the devices and methods described can be readily adapted to any number of other interventional procedures.

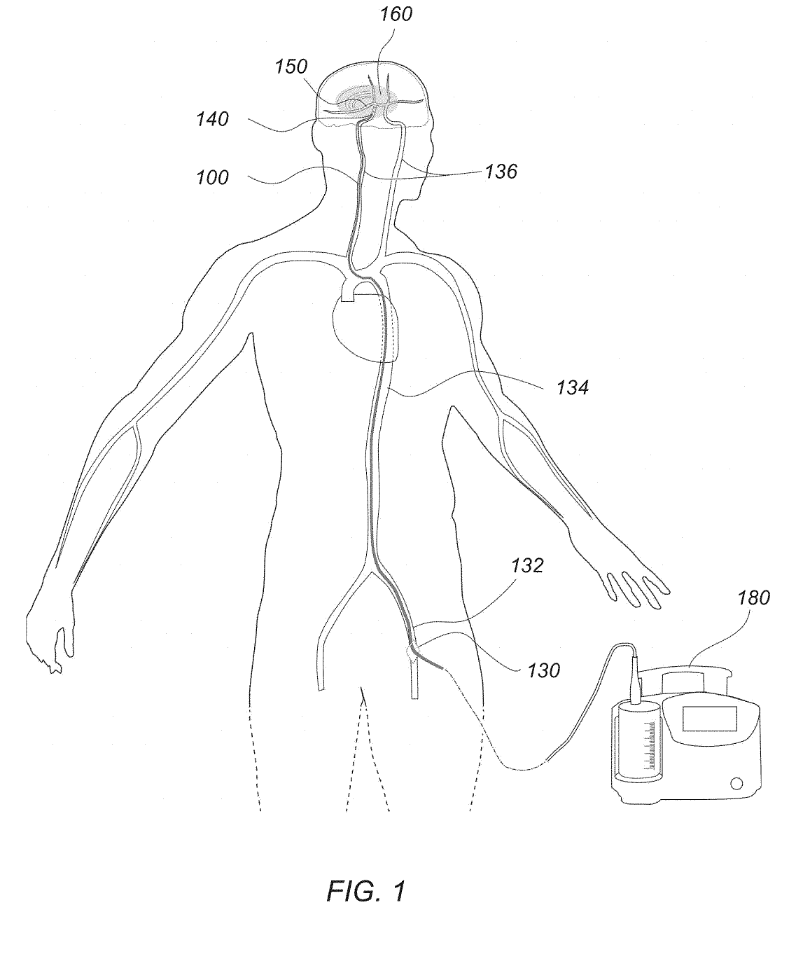

In treating a patient who has undergone a stroke, a neurosurgeon or interventional neuroradiologist may guide a balloon catheter 100 from, e.g., an incision 130 in the femoral artery 132 and through the blood vessels of the heart (such as the aorta 134) and neck (such as the carotid artery 136) and to a position in proximity of the infarcted region of the brain 160 adjacent to the clot 150, e.g., within 3 cm of the clot 150. The balloon suction catheter 100, as described in detail herein, may have an outer diameter suitable for advancement through the cerebrovasculature (e.g., 3 to 8 Fr) and may incorporate a balloon 140 and may also be fluidly coupled to a vacuum source 180, as illustrated in the example of FIG. 1.

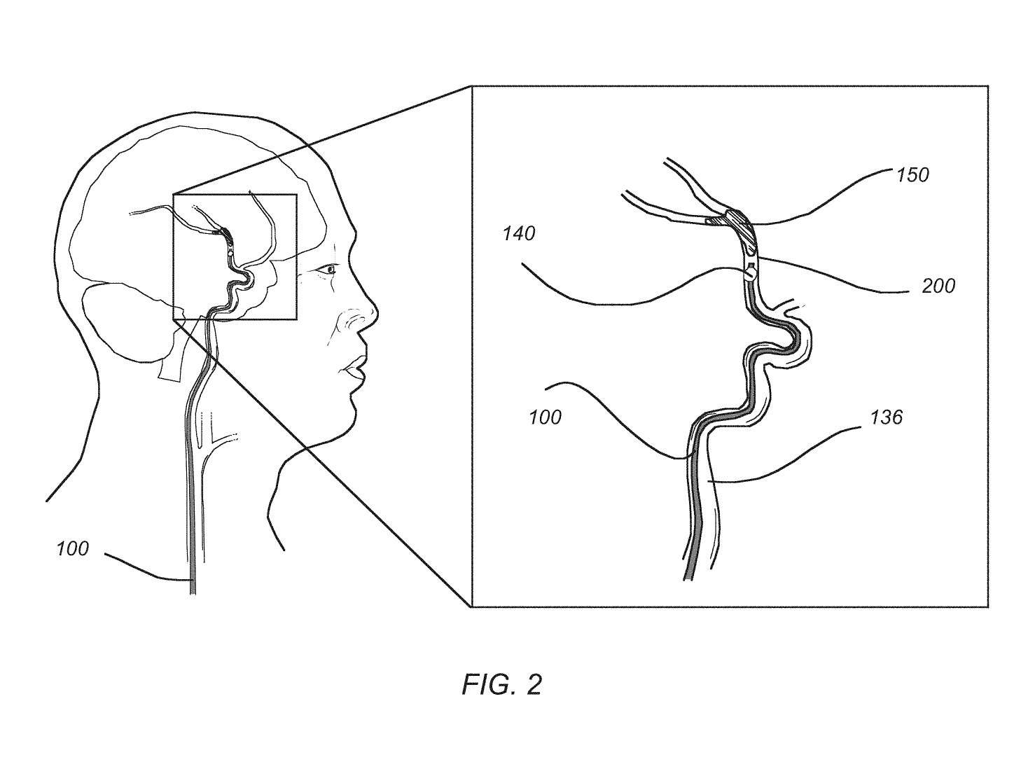

FIG. 2 shows a detail illustration of how the catheter 100 may be advanced through, e.g., the carotid artery 136 such that the balloon 140 and distal end of the catheter 100 is positioned into proximity of the clot 150. In this example, the clot 150 is illustrated as being lodged within and accessed through, e.g., the middle cerebral artery 200, although the catheter and methods of its use are not so limited to use in particular vasculature.

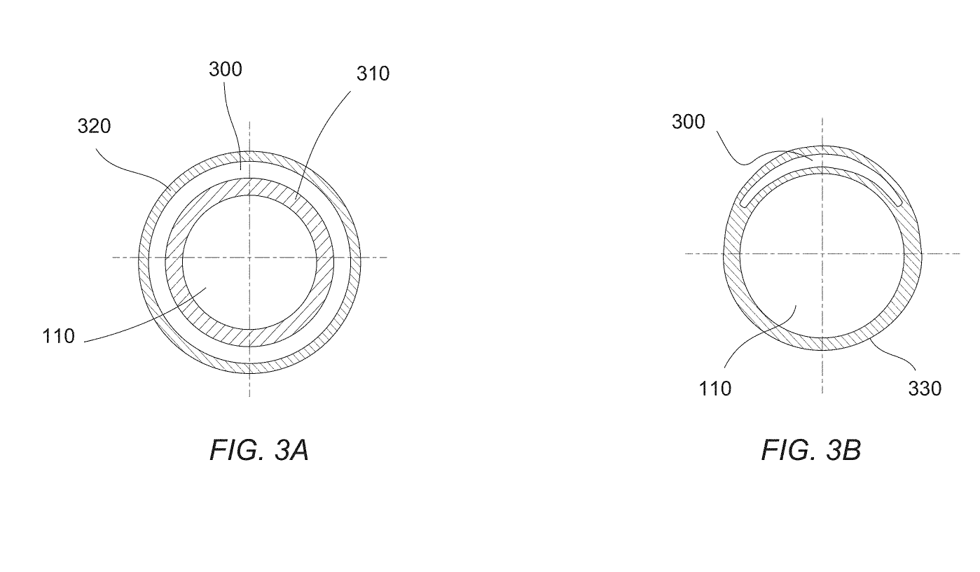

FIGS. 3A and 3B illustrate cross-sectional end views of conventional catheters where the fluid lumen 300 may be formed, e.g., between the outer wall of a primary lumen 310 and an inner wall of a secondary lumen 320, as shown in FIG. 3A. In such a configuration, the wall thicknesses of the primary lumen 310 and secondary lumen 320 may each range, e.g., between 0.003 to 0.007 in., forming an annular fluid lumen 300 having a space of, e.g., 0.004 to 0.015 in. The lumens may be fabricated from any number of biocompatible materials, e.g., nylon, Polyether Block Amides (PEBA), urethanes, etc.

Alternatively, the fluid lumen 300 may be formed within the wall of a multi-lumen extrusion 330, as shown in FIG. 3B. In such a configuration, the wall thickness of the extrusion 330 may range, e.g., between 0.003 to 0.010 in., and the fluid lumen 300 may have a width of, e.g., 0.002 to 0.015 in. The extrusion itself may be fabricated from various biocompatible materials, e.g., nylon, Polyether Block Amides (PEBA), urethanes, etc.

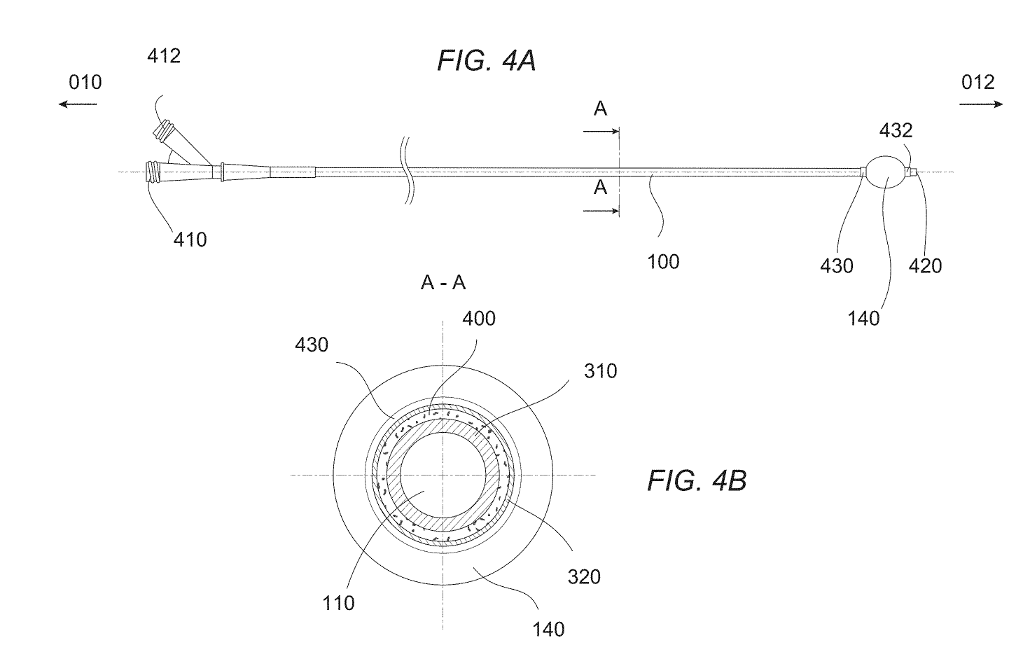

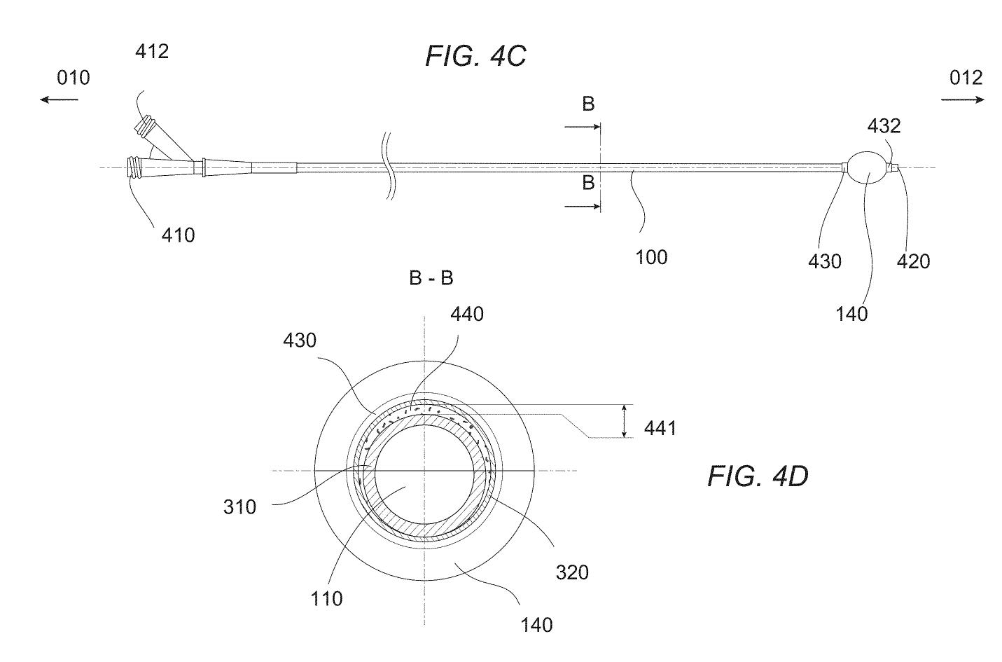

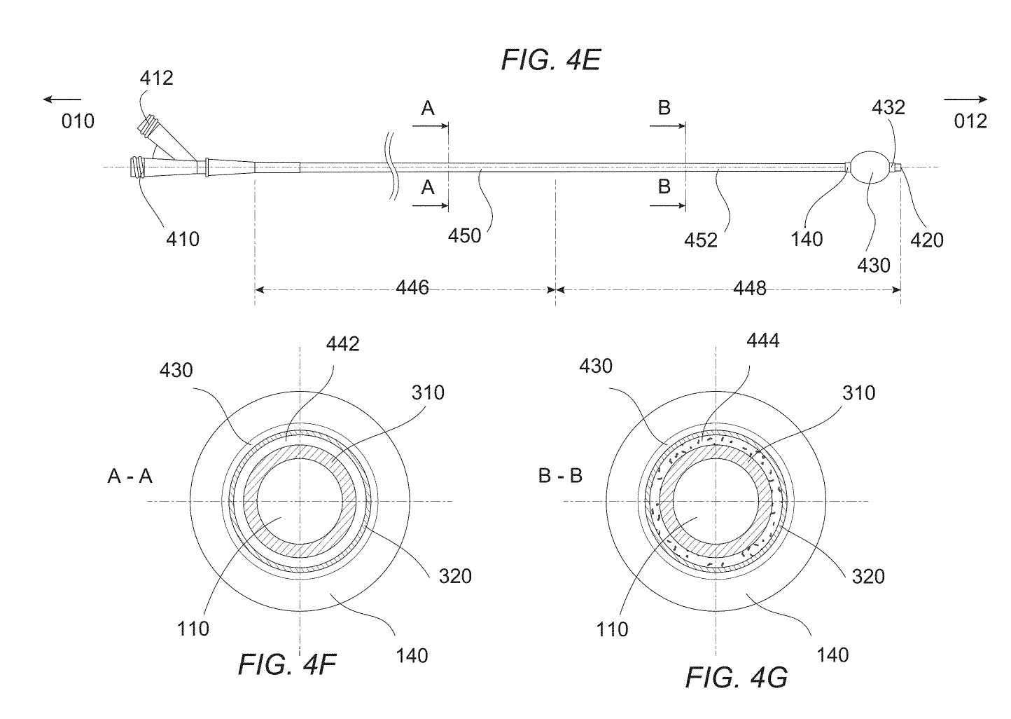

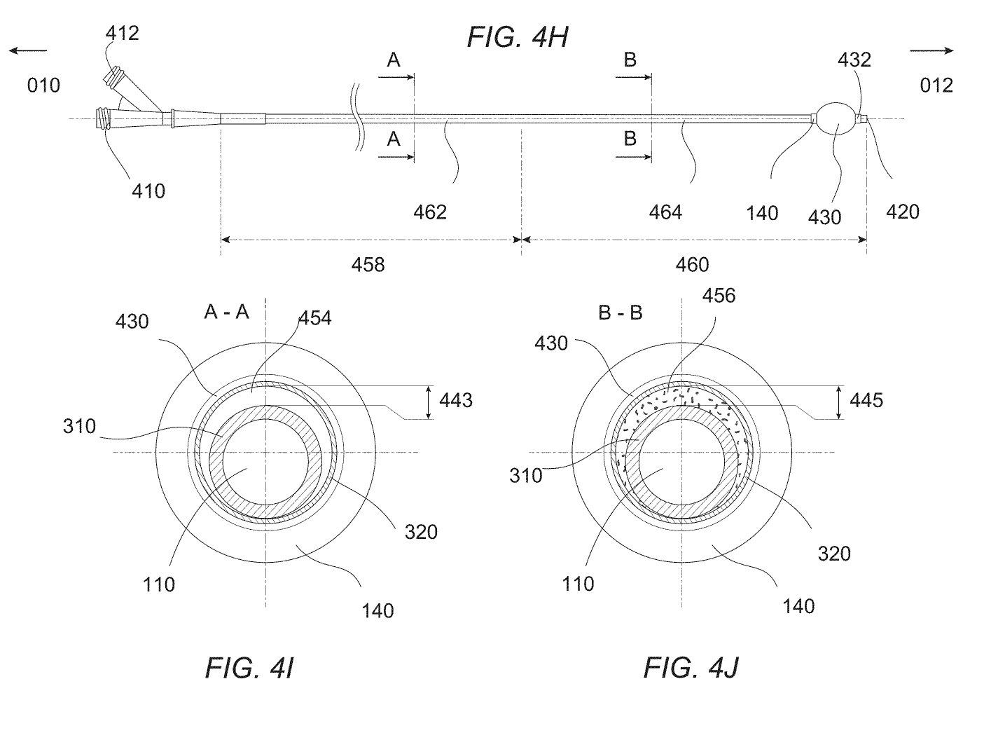

However, the conventional configurations shown in FIGS. 3A and 3B are subject to kinking and/or collapse when the catheter is placed under forces (e.g., push, torque, etc.) in challenging anatomies. As a result, the fluid lumens are subject to collapse preventing the delivery or suction of fluids through the lumen. FIGS. 4A and 4B illustrate side and cross-sectional end views of one embodiment of a catheter which prevents the fluid lumen from collapsing despite the forces imparted upon the catheter.

FIG. 4A shows a catheter 100 (e.g., PTFE lined 0.00025 to 0.00200 in. having a thickness preferably 0.00075 in.) having the inflation balloon 140 attached to the catheter 100 having a length of, e.g., 100 to 170 cm and preferably 130 cm. An inner diameter may be, e.g., between 0.0065 and 0.0080 in., and a primary wall may be comprised of, e.g., 3 to 16 segments of different durometer extrusions (about 80 D proximally to 70 A distally). The catheter body may be comprised of round or flat wire coil and/or braids reinforcement fabricated from, e.g., NiTi, stainless steel wire, etc. In one variation, the catheter body may be comprised of a mixed flat stainless steel and flat NiTi wire braid, e.g., 0.002 in. height×0.003 in. width for 80 to 100 cm along a proximal section then flat NiTi wire braid, e.g., 0.002 in. height×0.0015 in. width. The secondary lumen may be formed from a thin extrusion, e.g., 0.0005 to 0.0020 in. wall thickness, or 0.0005 to 0.0015 in. polyolefin.

The proximal end 10 of the catheter 100 may be coupled to a Luer hub having, e.g., a primary lumen 410 and a secondary lumen 412, fluidly coupled to the catheter 100 interior. The distal end 12 of the catheter 100 may have the inflation balloon 140 attached via a proximal neck of the balloon 430 and a distal neck of the balloon 432 such that a distal end of the primary lumen 420 extends beyond the balloon 140.

The cross-sectional view of FIG. 4B illustrates how a permeable structural interface 400 may be placed between the outer wall of the primary lumen 310 and an inner wall of the secondary lumen 320. The permeable structural interface 400 may be comprised of a number of various biocompatible materials which are configured to be permeable to fluids. This permeable structural interface 400 forms a preamble structural interface along the length of the catheter 100 and may be fully permeable to allow for the passage of fluids through the interface material. Yet because the interface 400 is also structurally robust, it may provide structural support between the lumens 310 and 320 such that the lumen spacing is maintained and resists collapse even when the catheter is kinked or torqued.

FIGS. 4C and 4D illustrate side and detailed cross-sectional end views of another embodiment of catheter 100 having a non-axisymmetric shape for the permeable interface. As shown in FIG. 4D, the transversal cross-section is shown having an arcuate-shaped orthogonal cross-section with respect to the axis of the catheter's primary lumen in plane B-B instead of an annular shape as described in FIGS. 4A and 4B. One of the advantages of the arcuate permeable interface is that for given outer and inner diameters of the permeable interface 440, the maximum height 441 of the permeable interface 440, defined by the gap between the walls of the primary lumen 310 and the secondary lumen 320 may be twice as large as the coaxial architecture described in FIGS. 4A and 4B.

In one variation, while a first gap is formed on a first side between the outer and inner diameters of the permeable interface 440, a second side between the outer and inner diameters of the permeable interface 440 may directly contact one another as illustrated in FIG. 4D. Alternatively, a second gap opposite to the first gap and which is also smaller than the first gap may be defined rather than having direct contact between the outer and inner diameters.

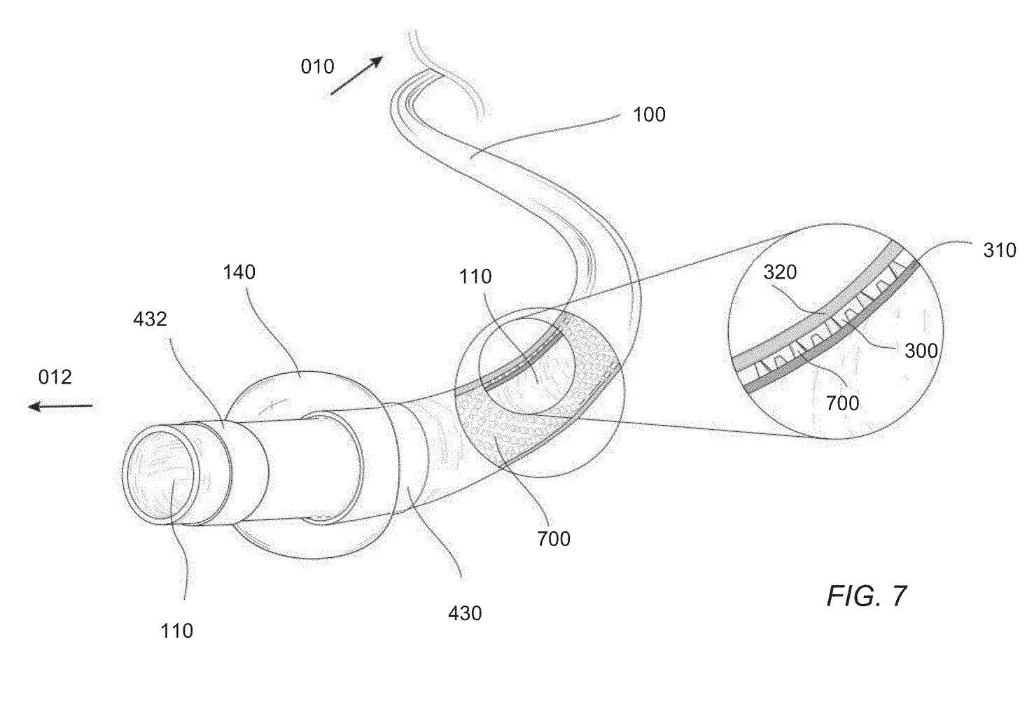

While the height of the permeable interface 440 may vary, a desirable maximum height may range, e.g., between 0.005 in to 0.020 in. Additionally, the permeable interface 440 may be comprised of, but is not limited to, micro-pillars such as micro-pillars 700 described below in FIG. 7, foams, grooves imprinted onto the primary lumen's outer surface, meshes, etc.

FIGS. 4E to 4G show yet another embodiment in the side and cross-sectional end views of a hybrid architecture comprised of a first part 450 of length 446 having coaxial cannulas with an inner cannula 310 and an outer cannula 320 defining a secondary lumen 442 between. This secondary lumen 442 may have an annular-shaped orthogonal cross-section with respect to the axis of the catheter's primary lumen as shown in plane A-A, and a second part 452 of length 448 constructed with an annular-shaped orthogonal cross-section permeable interface 444 as shown in in plane B-B. FIG. 4F illustrates a transversal cross-section of an embodiment of with the coaxial architecture defining an annular-shaped orthogonal cross-section with respect to the axis of the catheter's primary lumen.

While the height of the secondary lumen 442 defined between the inner cannula 310 and outer cannula 320 may vary, the height may range, e.g., between 0.003 in to 0.015 in. The thickness of the permeable interface 444 shown along the length 448 may vary as well, e.g., between 0.003 in. to 0.010 in. The length 446 may be varied to range between, e.g., 60 cm and 150 cm, while the length 448 may be varied to range between, e.g., 5 cm and 50 cm. Furthermore, the permeable interface 444 can be, but is not limited to, micro-pillars such as 700 defined in FIG. 7 and with various shapes, e.g., portions of cones or spheres, foams, groves imprinted onto the primary lumen's outer surface, meshes, etc.

FIGS. 4H to 4J show yet another embodiment with a hybrid architecture comprised of a first part 462 having a length 458 having the inner cannula 310 and outer cannula 320 disposed such that they define a secondary lumen 454 with an arcuate orthogonal cross-section with respect to the axis of the catheter's primary lumen as shown in plane A-A. The second part 464 of length 460 may be constructed with an arcuate permeable interface 456 of arcuate orthogonal cross-section with respect to the axis of the catheter's primary lumen as shown in plane B-B as disposed between inner cannula 310 and outer cannula 320.

FIG. 41 shows a transversal cross-section of the first part 462 having an arcuate secondary lumen 454 as shown in plane A-A. The height 443 represents the maximum distance between two points of the secondary lumen 454 both on a line of plane A-A passing through the center of the wall of the secondary lumen 320. FIG. 4J shows a transversal cross-section of the second part 462 having an arcuate permeable interface 456 as shown in plane B-B. The height 445 represents the maximum distance between two points of the secondary lumen 456 both on a line of plane B-B passing through the center of the wall of the secondary lumen 320.

While the height of the secondary lumen 320 defined between the inner cannula 310 and outer cannula 320 may vary, the height 443 of the secondary lumen may range, e.g., between 0.005 in to 0.020 in, and the height 445 of the permeable interface 456 may range, e.g., between 0.005 in to 0.020 in. The length 458 may range, e.g., between 60 cm and 150 cm, and the length 460 may range, e.g., between 5 cm and 50 cm. Furthermore, the permeable interface can be, but is not limited to, micro-pillars 700 defined in FIG. 7 and with various shapes, e.g., portions of cones or spheres, foams, grooves imprinted onto the primary lumen's outer surface, meshes, etc.

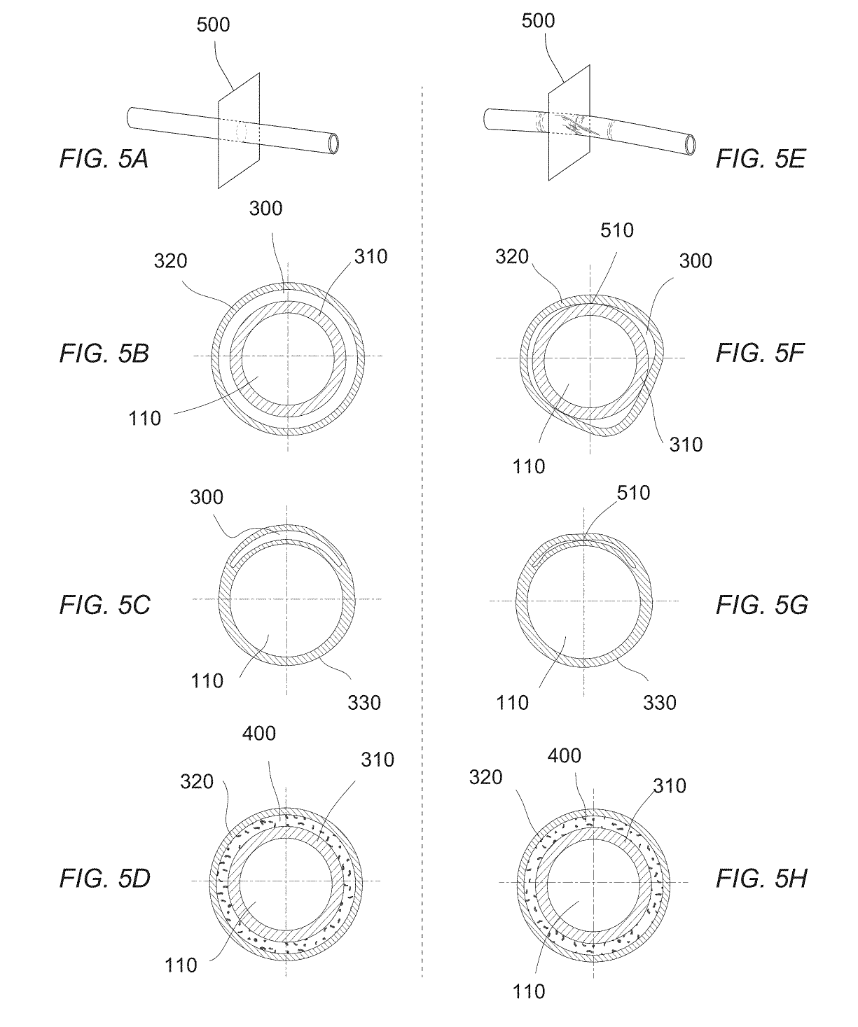

FIGS. 5A and 5E illustrate detail perspective views of a catheter in a straightened configuration and a bent or torqued configuration for comparison purposes to highlight possible failures, e.g., closure or collapse of the fluid lumen, of the conventional designs during bending or torquing while operating the balloon. One example is illustrated in FIGS. 5B and 5F which cross-sectional end views of a catheter along a plane 500 and having a primary lumen 310 and a secondary lumen 320 which form the fluid lumen 300 in-between when in a straightened configuration, shown in FIG. 5B, and collapsed configuration, shown in FIG. 5F. As indicated, contact 510 occurs between the lumens 310, 320 when the catheter 100 is bent or torqued impairing the inflation or deflation of the balloon catheter or impairing the delivery or suctioning of fluids and debris from the distal end of the catheter 100.

FIGS. 5C and 5G illustrate cross-sectional end views of a catheter having its fluid lumen 300 formed integrally within the lumen wall 330 when in a straightened and collapsed configuration for comparison purposes. As indicated in FIG. 5G, the fluid lumen 300 may contact 510 or collapse upon itself when the catheter is bent or torqued.

In contrast, FIGS. 5D and 5H illustrate cross-sectional end views of a catheter having a permeable preamble structural interface 400 positioned between the primary and secondary lumens 310, 320. The interface 400 is able to maintain the integrity of the fluid lumen 300 despite the catheter being bent or torqued.

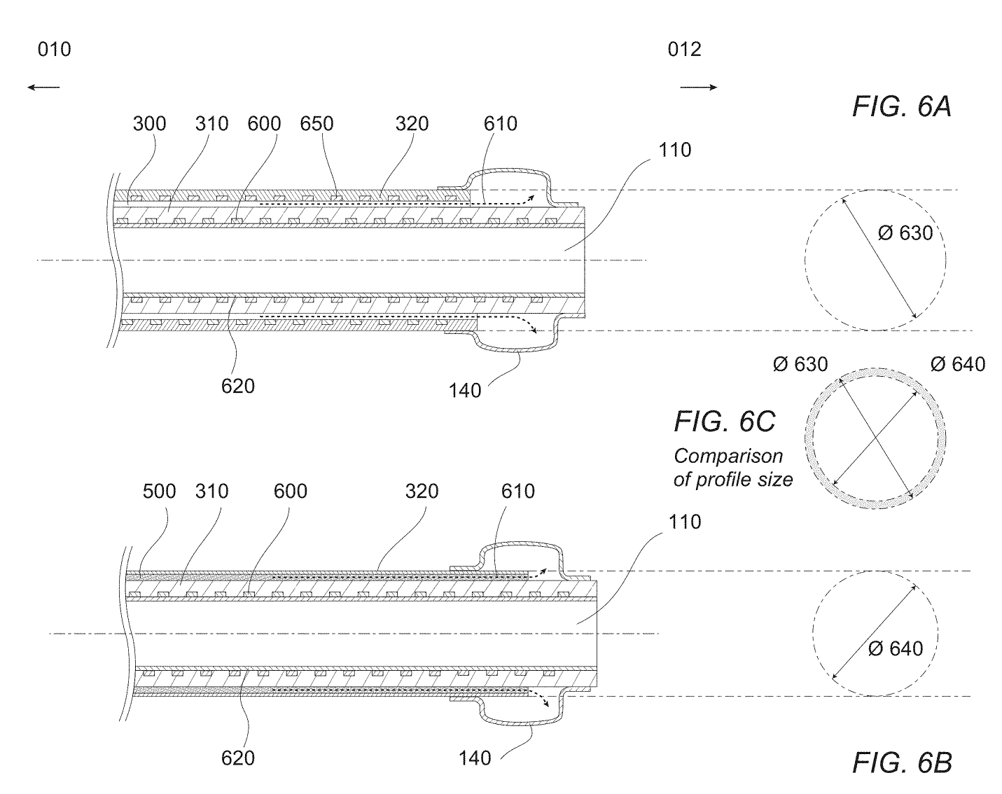

In another aspect of utilizing a preamble structural interface, the overall diameter of the catheter may be reduced relative to conventional catheter designs without sacrificing structural integrity. FIG. 6A illustrates an example of a conventional catheter having a first coil or braid reinforcement 600 around or within the wall of the primary lumen 310. A liner 620 may also be seen adjacent to the reinforcement 600. A second coil or braid reinforcement 650 is formed around or within the wall of the secondary lumen 320 to maintain the patency of the fluid path 610. The resulting first diameter 630 may be seen in the dashed end view.

In comparison, FIG. 6B illustrates a catheter having a preamble structural interface reinforcing the fluid lumen. As a result, the second coil or braid reinforcement 650 may be omitted entirely from the catheter to robustly operate the balloon. The resulting second diameter 640 may be seen in the dashed end view. As illustrated in FIG. 6C, the respective first diameter 630 of the conventional catheter and the second diameter 640 of the catheter having the structural interface are overlaid for comparison. The second diameter 640 illustrates a significant reduction in diameter from the first diameter 630 of the conventional catheter. Additionally, the absence of the second coil or braid reinforcement 650 allows for increased flexibility of the balloon catheter. In one example, the catheter of FIG. 6B may be formed from two reinforced coaxial extrusions having a total thickness of, e.g., 8 F for 6 F lumen.

In yet another embodiment, FIG. 7 illustrates a perspective and detail partial cross-sectional side view of an interface embodiment having an array of conical micro-pillars 700 for maintaining the fluid lumen. The array of micro-pillars 700 may be disposed on the outer surface of the primary lumen 310 and may act as a structural element to prevent the contact of the inner surface of the wall of the secondary lumen 320 and the outer surface of the wall of the primary lumen 310.

In one example, these micro-pillars 700 may have a height of, e.g., 0.001 to 0.006 in. or preferably 0.004 in. Moreover, the base of each individual micro-pillar 700 can take a variety of shapes, e.g., square, round, diamond-shape, etc. Furthermore, the micro-pillars 700 can be straight translations of the base or they may be angled compared to the outward normal vector to the catheter surface. The pillars 700 can also take on configurations such as pyramidal or conical with a base that is relatively larger than a top portion so that the Filling Ratio is reduced, where Filling Ratio is defined as follows:

$$Filling Ratio = \frac{Volume of Micro - Pillar Array}{Volume of Fluid Lumen} \qquad ( 1 )$$

The Volume of Fluid Lumen may be defined as the volume between the outer surface of the primary lumen and inner surface of the secondary lumen. Thus a fluid ratio may be maximized, where:

Fluid Ratio=1−Filling Ratio (2)

Additionally, the patterning of the micro-pillars may be arranged in various patters (e.g., square, staggered rows, etc. and as further described herein) where the spacing between the centers of adjacent pillars may range, e.g., between 0.005 to 0.040 in. The secondary lumen 320 may be made from, e.g., PEBAX, with a varying durometer starting from, e.g., 80 D proximally to 60 A distally, and having a wall thickness of, e.g., between 0.001 to 0.004 in. Alternatively, the secondary lumen may be fabricated from, e.g., shrink tube such as polyolefin, having a wall thickness of, e.g., between 0.0005 to 0.0020 in.

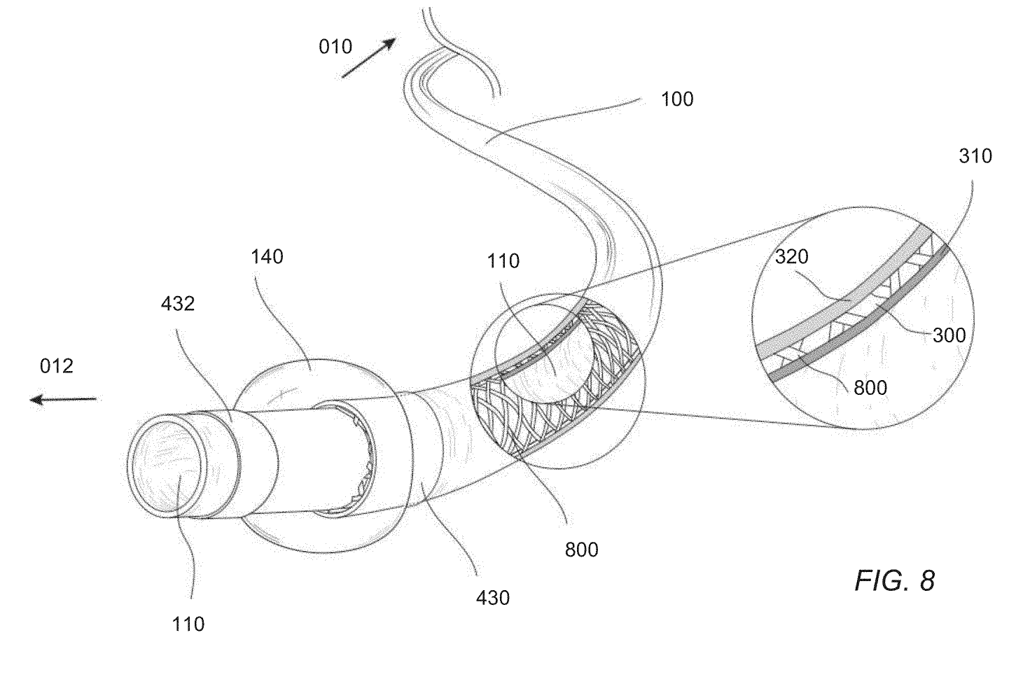

FIG. 8 illustrates a perspective and detail partial cross-sectional side view of an another interface embodiment having a braided interface 800 for maintaining the fluid lumen. The braid 800 may be fabricated from various materials, e.g., Nickel, Gold, Platinum, Tungsten, NiTi, Steel, polymers such as Nylon, etc. Moreover, the braids may be made from, e.g., round, flat wire or a combination of both to increase the Fluid Ratio. Additionally, the braids may be made from wire having a thickness of, e.g., 0.001 to 0.003 in., such that the overall thickness of the braid may range, e.g., between 0.002 to 0.006 in.

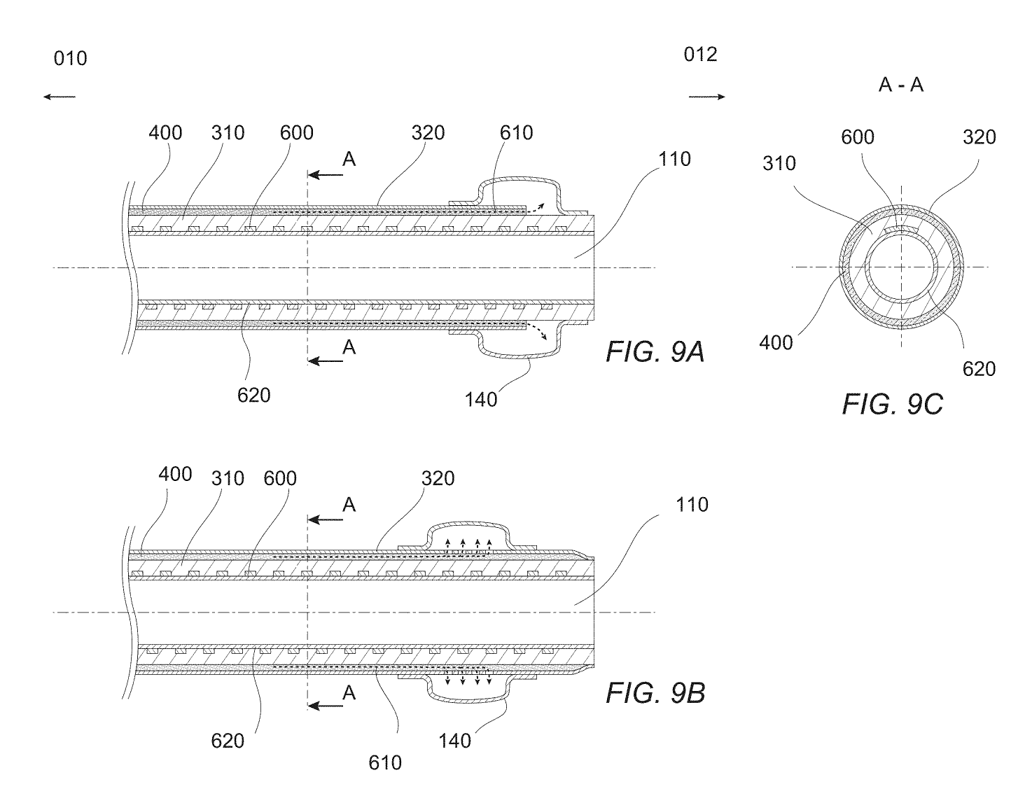

FIGS. 9A to 9C illustrate cross-sectional side and end views of various embodiments where the fluid lumen 610 may be open at a distal opening within the balloon 140, as shown in FIG. 9A, or along a side portion of the fluid lumen within the balloon 140, as shown in FIG. 9B. In the case where the fluid lumen 610 is opened at a distal opening, a proximal neck of the balloon 140 may be bonded onto the secondary lumen 320 while the distal neck of the balloon 140 may be bonded to the primary lumen 310. In the case where the fluid lumen is opened along a side portion, the secondary lumen 320 can also be bonded near the distal end of the primary lumen 310, e.g., 0 to 10 mm. One or more inflation holes each having a diameter of, e.g., 50 500 μm, may be drilled near the distal end of the secondary lumen 320 in various patterns, e.g., 3 staggered rows, 6 holes equally distributed radially. FIG. 9C illustrates a cross-sectional end view showing the arrangement of the primary lumen 310, secondary lumen 320, braid 600, and interface 400.

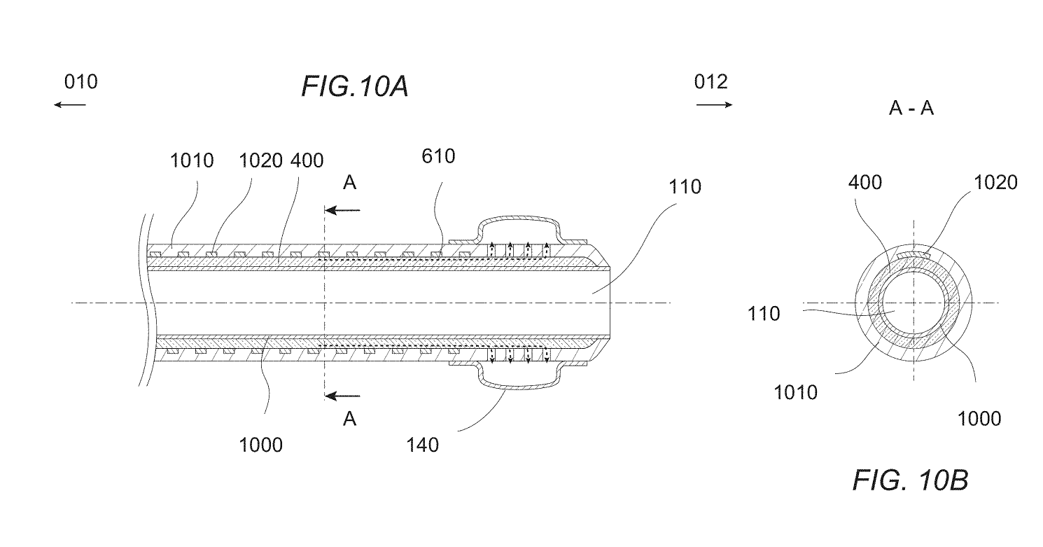

FIGS. 10A and 10B illustrate cross-sectional side and end views of an alternative arrangement of the lumen, permeable interface, and balloon 140. In this configuration, the permeable interface within the balloon 140 may be disposed between the liner 1000, which may function as the wall of the primary lumen and fabricated from, e.g., PTFE, and the secondary lumen 1010, which may be fabricated from, e.g., PEBAX. The coil or braid reinforcement 1020 for the secondary lumen 1010 may function as the structural component of the catheter itself.

The micro-pillars described herein are small bodies compared to the diameter of the catheter. They may be arranged on the surface of the generally cylindrical primary lumen and their function is to create a permeable structural interface between the wall of the primary lumen and the secondary lumen. The permeability of the micro-pillar interface allows for the movement of fluid in between the walls of the primary and secondary lumen and their mechanical properties prevent contact between the walls of the of the lumens.

The micro-pillar shapes may be comprised generally on volumes enclosed within the surface generated by translation of a contour (e.g., cylindrical solids, etc.) where the surface generated by the projection of the contour onto the primary lumen's wall and a plane are more distant from the axis of the primary lumen than its outer surface. The contours described are geometrical shapes drawn onto the tangential plane to the primary lumen's surface. In the examples described herein, direction of translation of the contour may be normal to the surface of the primary lumen's outer wall and applied to the centroid of the base, but the micro-pillars are not so limited and may have shapes with directions that are not normal to that surface.

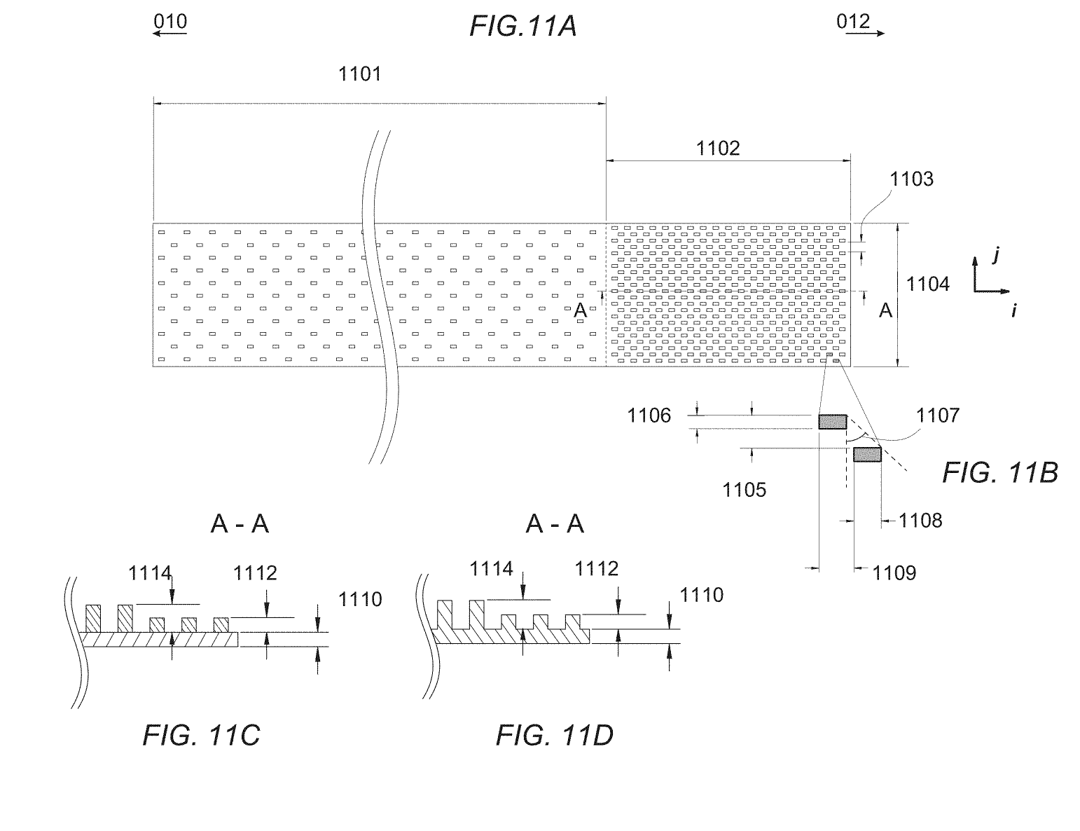

FIGS. 11A and 11B illustrate a top view of an outer surface of a primary lumen wall splayed out illustrating how the density of micro-pillars may be varied along a length of the interface. In this example, a first micro-pattern array may be disposed along a first proximal section 1101 of the lumen wall while a second micro-pattern array may be disposed along a second distal section 1102 of the lumen wall. The density of the micro-pattern arrays may be varied once, twice, or any number of times along the length of the lumen wall. For example, the surface density of the micro-pillars may be comprised of, e.g., between 3% and 30%, where the surface of micro-pillars is divided by surface of free space calculated on the unrolled model. The density of the micro-pillars may be comprised of, e.g., between 100 and 500 micro-pillars per square cm. Moreover, the micro-pillars can be aligned along the direction i (axial), in a staggered pattern or by following a different pattern described by multiple helices or a random order. The micro-pillars can be the same size and shape in the different sections or they may differ to offer a varying flexibility.

FIG. 11B illustrates a detail top view of micro-pillars to illustrate how various parameters may be varied depending on the desired characteristics of the catheter. For instance, parameters such as the distance 1103 between two adjacent micro-pillars around the perimeter 1104 of the primary lumen wall may be varied. Other parameters such as the distances 1105 between the micro-pillars of a unit cell along the j direction (circumferential) may be varied as are the distances 1109 along the i direction. Additionally, parameters such as the widths 1106 of the micro-pillar along the j direction and/or the widths 1108 along the i direction may also be varied. Other parameters such as the angle 1107 between the micro-pillars making a unit cell may also be varied as well.

FIGS. 11C and 11D illustrate detail side views of various configurations of the micro-pillar arrays having varying heights which may be formed, e.g., via additive or subtractive manufacturing processes. For instance, the thickness 1110 of the primary lumen without the micro-pillars is shown and the micro-pillars may have varying heights where a first array of micro-pillars have a first height 1112 and a second array of micro-pillars have a second height 1114 which is relatively higher than the first height 1112.

Any of the parameters (widths, lengths, angles, spacing, heights, etc.) and/or densities described may be combined in any number of combinations and are not limited. In one variation for neurovascular applications, the proximal section 1101 may have a length of, e.g., 60 to 120 cm and preferably 100 cm, which the distal section 1102 may have a length of, e.g., 5 to 70 cm and preferably 30 cm. The distance 1103 may range, e.g., between 0.003 and 0.020 in. and preferably 0.010 in. in the distal section 1102. The perimeter 1104 may range, e.g., between 0.050 and 0.090 in. and preferably 0.080 in.

The distance 1105 may range, e.g., between 0.006 and 0.030 in. distally and, e.g., between 0.0010 and 0.0800 in. proximally while the distance 1109 may range, e.g., between 0.010 and 0.040 in. distally and, e.g., between 0.020 and 0.100 in. proximally. The width 1106 may range, e.g., between 0.003 and 0.020 in. and preferably 0.005 in. in the distal section 1102 and the width 1108 may range, e.g., between 0.003 and 0.010 in. The angle 1107 may range, e.g., between 0 and 90 degrees and preferably 45 degrees.

With respect to the heights, the thickness 1110 may range, e.g., between 0.003 and 0.010 in. while the heights of the micro-arrays 1112 and 1114 may range, e.g., between 0.002 and 0.010 in.

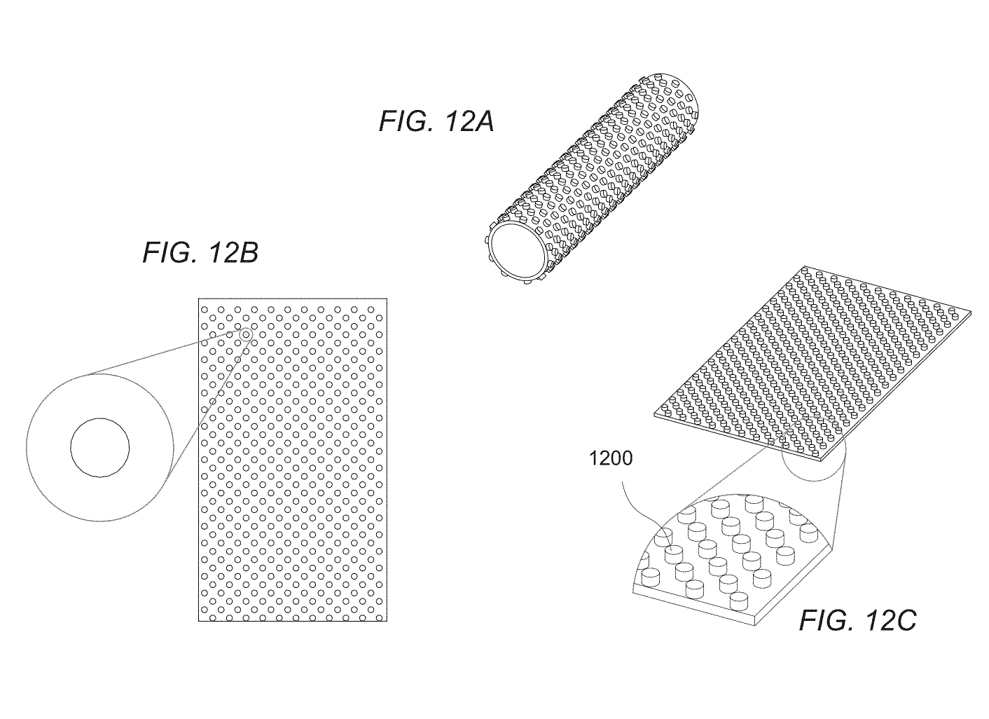

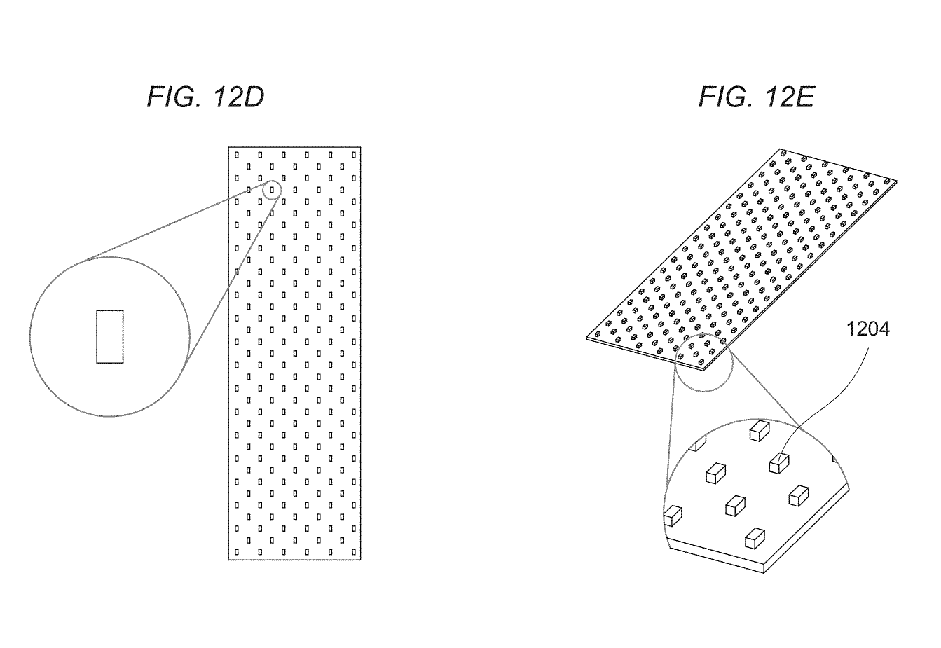

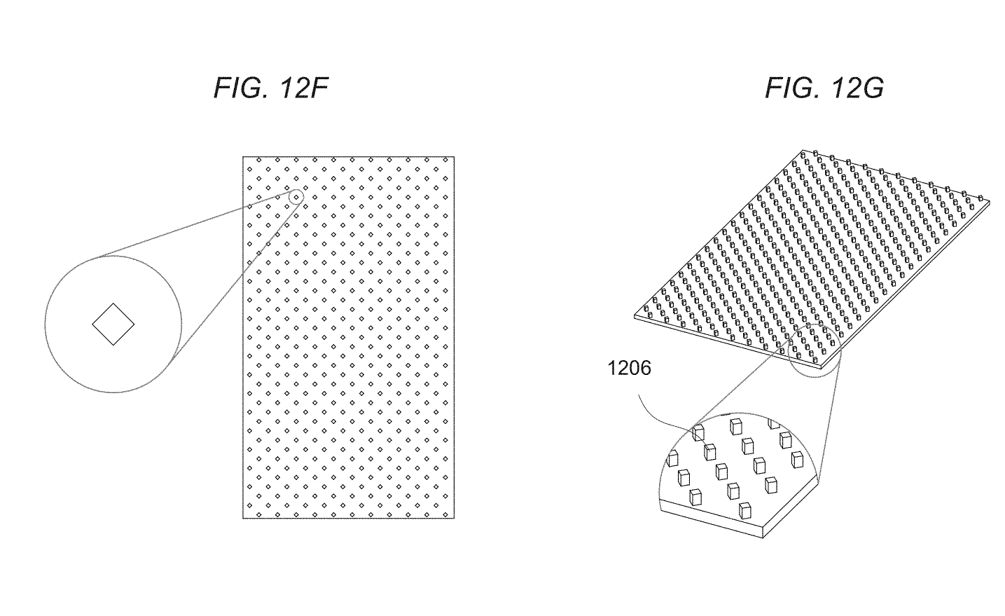

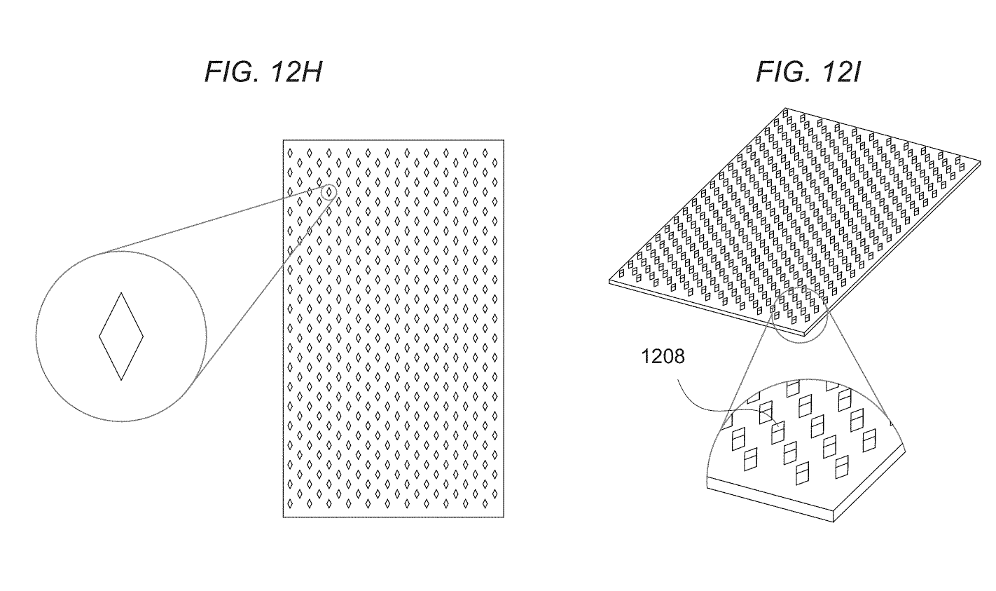

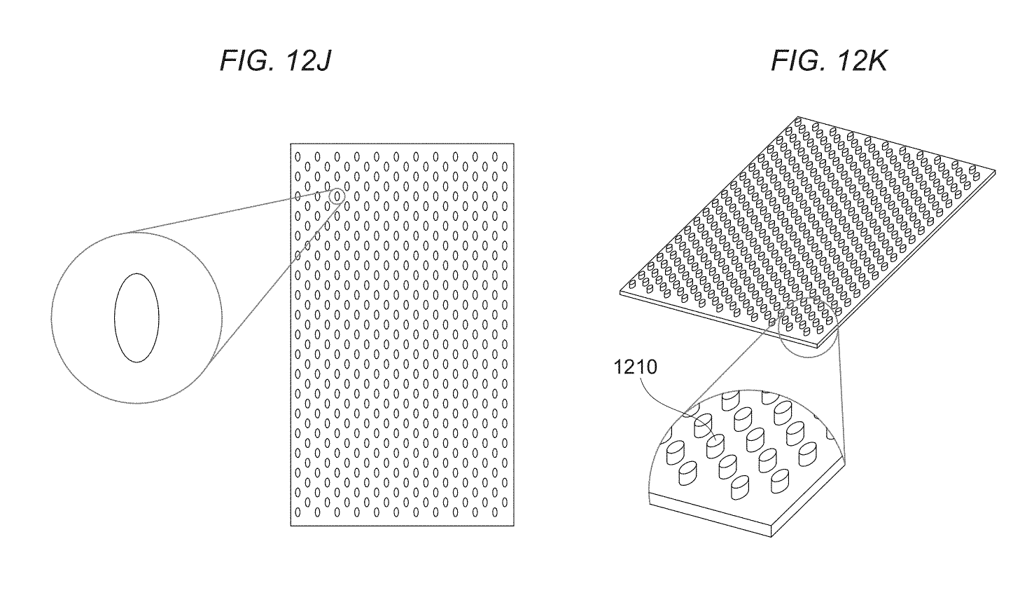

FIGS. 12A to 12C illustrate perspective and top views of yet another embodiment of the micro-pillar configuration as cylindrical structures formed upon the outer wall of the primary lumen (illustrated in top and perspective splayed orientations in FIGS. 12B and 12C). The micro-pillars 1200 are configured as circle-based cylindrical micro-pillar structures.

FIGS. 12D and 12E illustrate top and perspective splayed orientations of yet another embodiment of the micro-pillar 1204 configuration as rectangular structures formed upon the outer wall of the primary lumen.

FIGS. 12F and 12G illustrate top and perspective splayed orientations of yet another embodiment of the micro-pillar 1206 configuration as diamond-shaped structures formed upon the outer wall of the primary lumen.

FIGS. 12H and 12I illustrate top and perspective splayed orientations of yet another embodiment of the micro-pillar 1208 configuration as elongated diamond-shaped structures formed upon the outer wall of the primary lumen.

FIGS. 12J and 12K illustrate top and perspective splayed orientations of yet another embodiment of the micro-pillar 1210 configuration as elliptically-shaped structures formed upon the outer wall of the primary lumen.

The micro-pillars may alternatively be formed as full or partial cones where cones are understood to be volumes enclosed with the surface generated by the lines connecting a contour to a vertex. The surface generated by the projection of the contour onto the primary lumen's wall and a plane are more distant from the axis of the primary lumen than its outer surface. The vertices may be on a line normal to the contour that passes by its centroid. Partial cones may be formed by cones that are truncated and are delimited by a plane (e.g., tangential to the primary lumen's surface) that is less distant from the primary lumen's axis than the vertex.

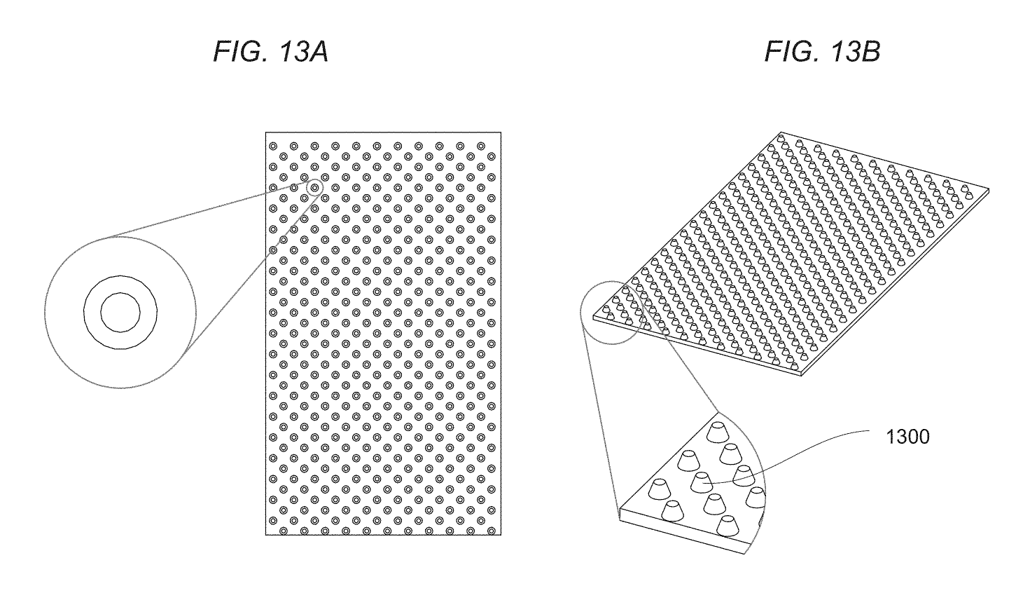

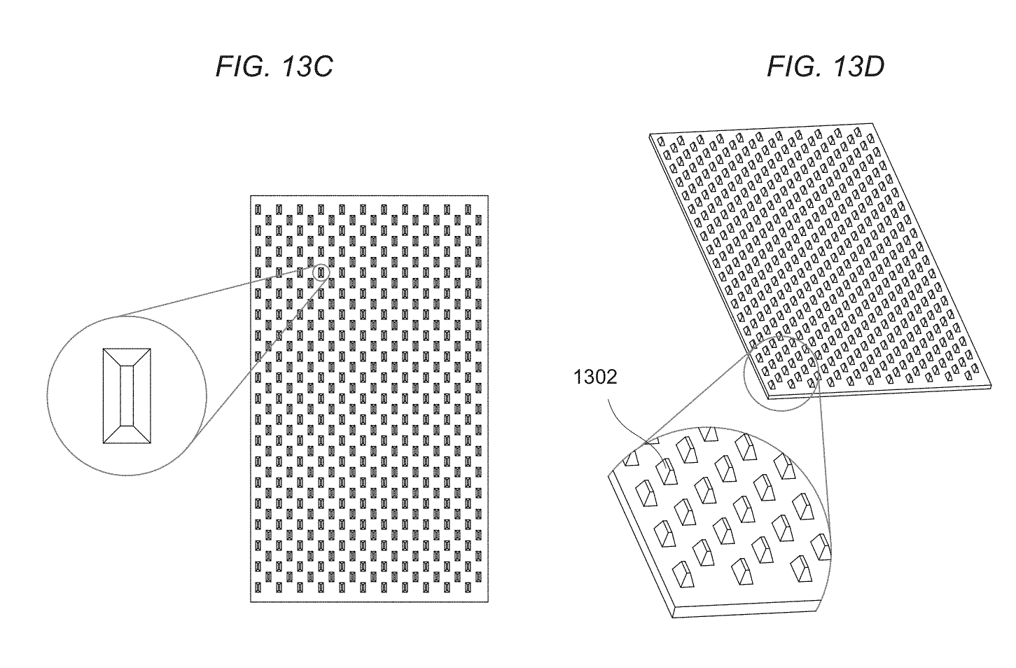

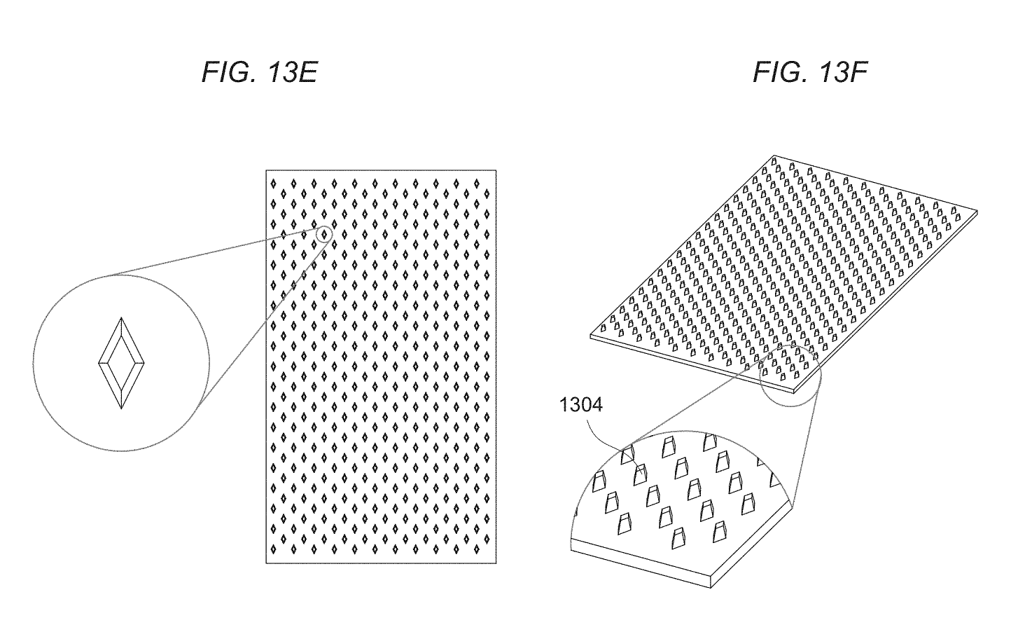

FIGS. 13A and 13B illustrate top and perspective splayed orientations of yet another embodiment of the micro-pillar 1300 configuration as conically-shaped structures formed upon the outer wall of the primary lumen.

FIGS. 13C and 13D illustrate top and perspective splayed orientations of yet another embodiment of the micro-pillar 1302 configuration as truncated pyramid-shaped structures formed upon the outer wall of the primary lumen.

FIGS. 13E and 13F illustrate top and perspective splayed orientations of yet another embodiment of the micro-pillar 1304 configuration as truncated diamond-shaped structures formed upon the outer wall of the primary lumen.

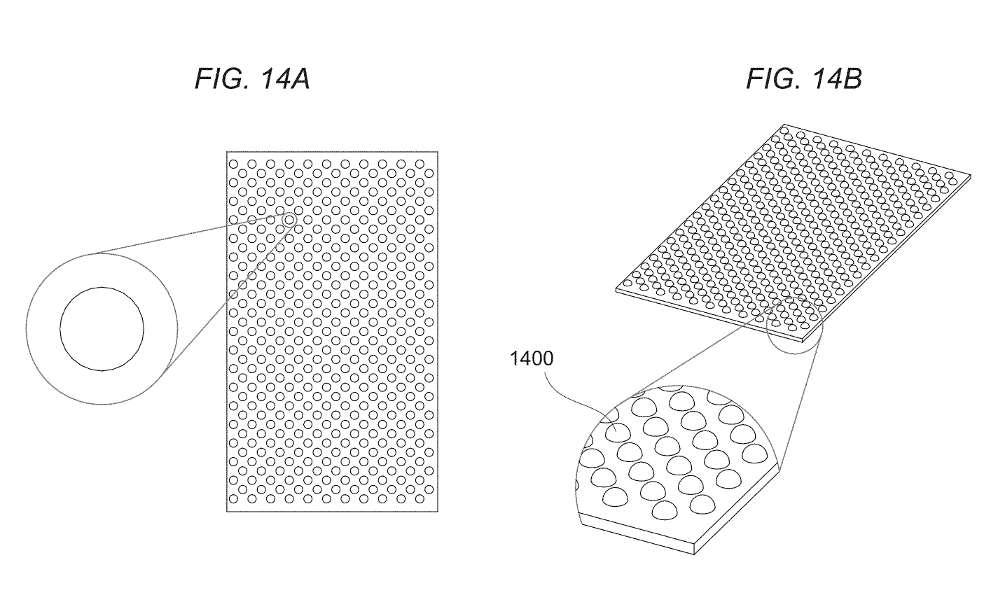

FIGS. 14A and 14B illustrate top and perspective splayed orientations of yet another embodiment of the micro-pillar 1400 configuration as hemispherically-shaped structures formed upon the outer wall of the primary lumen. Various other shapes, such as partial ellipsoids, partial paraboloids, etc., may be utilized in other variations.

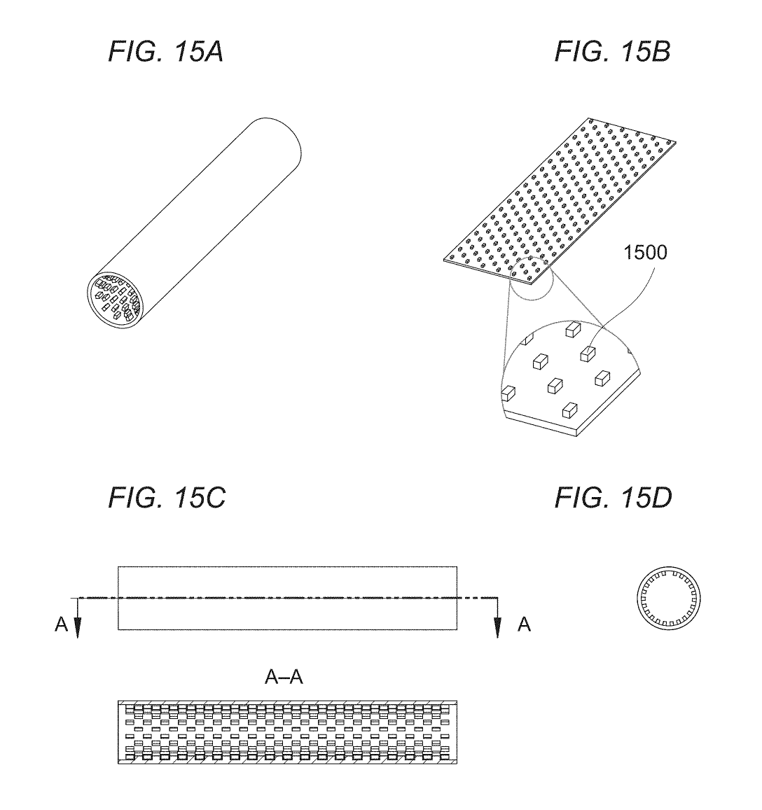

Alternatively, rather than having the micro-pillars disposed upon the outer wall of the primary lumen, they may instead be disposed upon the inner wall of the secondary lumen, as shown in the perspective view of FIG. 15A. FIGS. 15B and 15C show perspective splayed and cross-sectional views of the secondary lumen and FIG. 15D shows an end view.

In this and any of the other variations described, the micro-pillars may be formed on the outer wall of the primary lumen, the inner wall of the secondary lumen, or a combination of both. Moreover, any of the micro-pillar embodiments may be used in any combination with the outer wall of the primary lumen, the inner wall of the secondary lumen, or both.

The applications of the devices and methods discussed above are not limited to applications within the cerebrovasculature but may include any number of further treatment applications such as those used in interventional radiology and cardiology, interventional peripheral radiology, interventional pulmonology, interventional nephrology, peripheral artery disease, deep vein thrombosis, pulmonary embolism, abdominal aortic aneurysms, acute limb ischemia, etc. Moreover, such devices and methods may be applied to other treatment sites within the body. Modification of the above-described assemblies and methods for carrying out the invention, combinations between different variations as practicable, and variations of aspects of the invention that are obvious to those of skill in the art are intended to be within the scope of the claims.

What is claimed is:

1. A catheter configured to maintain patency of a fluid lumen, comprising:
a catheter body having at least one fluid lumen defined therethrough;
a structural interface disposed within the at least one fluid lumen, wherein the structural interface is configured to be permeable to fluids and further has a structural integrity which is sufficient so as to inhibit contact between walls of the fluid lumen such that patency of the fluid lumen is maintained.

2. The catheter of claim 1 wherein the fluid lumen is defined within a wall of the catheter body.

3. The catheter of claim 1 wherein the catheter is comprised of a primary lumen and a secondary lumen coaxially aligned about the primary lumen and forming the fluid lumen therebetween.

4. The catheter of claim 3 wherein the structural interface is comprised of a braid positioned between the primary lumen and the secondary lumen.

5. The catheter of claim 1 wherein the structural interface is comprised of an array of micro-pillars extending between an outer wall of the primary lumen and an inner wall of the secondary lumen.

6. The catheter of claim 5 wherein the array of micro-pillars is attached to the outer wall of the primary lumen.

7. The catheter of claim 5 wherein the array of micro-pillars is attached to the inner wall of the secondary lumen.

8. The catheter of claim 5 wherein the array of micro-pillars has a first spacing density along a first portion of the catheter and a second spacing density along a second portion of the catheter.

9. The catheter of claim 5 wherein the array of micro-pillars has a non-uniform spacing density.

10. The catheter of claim 5 wherein the array of micro-pillars has a first set of micro-pillars having a first height and a second set of micro-pillars having a second height which is different from the first height.

11. The catheter of claim 5 wherein the array of micro-pillars is configured to have a shape which is cylindrical, rectangular, diamond-shaped, elongated diamond-shaped, elliptical, conical, pyramidal, or hemispherical.

12. The catheter of claim 3 wherein the structural interface comprises one or more grooves defined along an outer surface of the primary lumen.

13. The catheter of claim 1 wherein the fluid lumen defines a cross-section having an arcuate shape.

14. The catheter of claim 1 wherein the catheter body comprises a first proximal portion without the structural interface and a second distal portion having the structural interface.

15. The catheter of claim 14 wherein the first proximal portion comprises a primary lumen and a secondary lumen coaxially aligned about the primary lumen and forming the fluid lumen therebetween.

16. The catheter of claim 14 wherein the second distal portion defines an annular orthogonal cross-section.

17. The catheter of claim 14 wherein the fluid lumen along the first proximal portion has an arcuate shape.

18. The catheter of claim 14 wherein the structural interface along the second distal portion has an arcuate shape.

19. A catheter configured to maintain patency of a fluid lumen, comprising:
a catheter body having a primary lumen and a secondary lumen coaxially aligned about the primary lumen and forming a fluid lumen therebetween;
an array of micro-pillars disposed within the fluid lumen at a predetermined density and configuration and extending between an outer wall of the primary lumen and an inner wall of the secondary lumen,
wherein the array of micro-pillars is configured to be permeable to fluids and further has a structural integrity which is sufficient so as to inhibit contact between walls of the fluid lumen such that patency of the fluid lumen is maintained.

20. The catheter of claim 19 wherein the array of micro-pillars is attached to the outer wall of the primary lumen.

21. The catheter of claim 19 wherein the array of micro-pillars is attached to the inner wall of the secondary lumen.

22. The catheter of claim 19 wherein the array of micro-pillars has a first spacing density along a first portion of the catheter and a second spacing density along a second portion of the catheter.

23. The catheter of claim 19 wherein the array of micro-pillars has a non-uniform spacing density.

24. The catheter of claim 19 wherein the array of micro-pillars has a first set of micro-pillars having a first height and a second set of micro-pillars having a second height which is different from the first height.

25. The catheter of claim 19 wherein the array of micro-pillars is configured to have a shape which is cylindrical, rectangular, diamond-shaped, elongated diamond-shaped, elliptical, conical, pyramidal, or hemispherical.

26. The catheter of claim 19 wherein the fluid lumen defines a cross-section having an arcuate shape.

27. The catheter of claim 19 wherein the catheter body comprises a first proximal portion without the array and a second distal portion having the array.

28. The catheter of claim 27 wherein the fluid lumen along the first proximal portion has an arcuate shape.

29. The catheter of claim 27 wherein the fluid lumen along the second distal portion has an arcuate shape.

30. A method of maintaining lumen patency, comprising:
providing a catheter body having a primary lumen and a secondary lumen coaxially aligned about the primary lumen and forming a fluid lumen therebetween; and
forming an array of micro-pillars disposed within the fluid lumen at a predetermined density and configuration and extending between an outer wall of the primary lumen and an inner wall of the secondary lumen,
wherein the array of micro-pillars is configured to be permeable to fluids and further has a structural integrity which is sufficient so as to inhibit contact between walls of the fluid lumen such that patency of the fluid lumen is maintained.
